(19) United States
(12) Patent Application Publication
(10) Pub. No.: US 2015/0119888 A1
(43) Pub. Date: Apr. 30, 2015

(54) TENSIONING DEVICE FOR BONE CERCLAGES

(71) Applicant: Königsee Implantate GmbH, Allendorf OT Aschau (DE)

(72) Inventors: Knut Hentschel, Rudolstadt (DE); Matthias Bock, Konigsee-Rottenbach (DE); Stefan Hegermann, Kempen/Niederrhein (DE); Sven Pfeiffer, Konigsee-Rottenbach (DE); Franz Rennebaum, Wuppertal (DE); Christian Günsche, Herschdorf (DE)

(21) Appl. No.: 14/527,296

(22) Filed: Oct. 29, 2014

(30) Foreign Application Priority Data
Oct. 31, 2013 (DE) 102013018346.4

(51) Int. Cl. A61B 17/88

(52) U.S. Cl. CPC A61B 17/8869
USPC 606/74

(57) ABSTRACT

The invention relates to a tensioning device for bone cerclages, consisting of a guide part with a spindle located in the interior which receives a slide secured against rotation relative to the guide part, wherein the spindle is operatively connected to a rotary handle such that the slide is adjustable inside the guide part in the direction of the longitudinal axis of same, and further including means for the clamped attachment of the bone cerclage on the slide. According to the invention the means for clamping comprise a preferably two-part assembly group formed of an upper and a lower clamping piece, wherein the upper and the lower clamping pieces are connected to each other by a hinged joint so as to be foldable, and on the side opposite the hinged joint a clamping screw is provided to exert a force on the clamping pieces and fix the bone cerclage located between the clamping pieces in position, the surfaces of the clamping pieces pointing to the bone cerclage having clamping structures that permit the fixing of rope, wire and band bone cerclages in position.

The invention relates to a tensioning device for bone cerclages, consisting of a guide part with a spindle located in the interior which receives a slide secured against rotation relative to the guide part, wherein the spindle is operatively connected to a rotary handle such that the slide is adjustable inside the guide part in the direction of the longitudinal axis of same, and further including means for the clamped attachment of the bone cerclage on the slide, according to the preamble of claim 1.

So-called bone cerclages are known for fixing bone fragments with each other or against each other, and/or for attaching bone plates.

A bone plate is tensioned with respect to the bone, for instance, using a fixing wire. To this end, a wire tensioning guide is used. The tensioning step is followed by twisting and cutting through the wire cerclage.

As opposed to wire cerclages, cerclage bands having a broader contact surface of the wire band allow a more favorable pressure distribution due to the larger surface area. The cerclage is thus prevented from cutting into the bone. Collets are used for tensioning cerclage bands, whereby the actual bone cerclage is fixed by a lock.

Further known is a cerclage system, offered and distributed by the company GP-Medical, Lautertal, Germany. This cerclage system firstly places the cerclage band around the bone. The cerclage band is then threaded into the cerclage tensioner and a guide hole provided there, and is bent in an S-shaped manner. The tensioning device has a spindle drive which is capable of effecting a longitudinal shifting motion of a slide mounted in a guide part. The slide takes along the end of the band cerclage, which is bent in an S-shaped manner. If the band cerclage begins to move out of the S-shape, it will be assumed that the tension between the cerclage band and the bone, respectively, bone and bone plate is sufficient. The process of tensioning is then stopped, and the band is secured by bending it at right angles and cutting off the projecting end of the band later.

As can be seen from the above explanations, the configuration of tensioning devices for wire cerclages differs from those for band cerclages. It is hence necessary that surgical wards in hospitals have different tensioning devices available. If a first tension, e.g. accomplished by means of a wire cerclage, proves to be unsatisfactory, the change to a band cerclage requires the exchange of the complete equipment, and this is intraoperatively disadvantageous.

Based on the foregoing it is, therefore, the object of the invention to provide an improved tensioning device, which includes a spindle drive, and is capable of tensioning the most various bone cerclages, viz. wire cerclages consisting of wires or ropes of different diameters, or also band cerclages, without a need for particular manipulation steps or the exchange of instruments.

The solution to the object of the invention is achieved by the tensioning device according to the combination of features recited in claim 1. The dependent claims define at least useful embodiments and further developments.

Accordingly, there is proposed a tensioning device for bone cerclages, which consists of a guide part with a spindle located in the interior. The guide part may be designed as a guide tube or other hollow profile.

A spindle is provided in the interior of the guide part which receives a slide secured against rotation relative to the guide part. The rotation protection may be realized by the cross-section of the slide having a shape different from a circular shape and being adapted, in this regard, to the inner cross-section of the guide part. If the guide part has a purely tubular configuration the slide can be mounted in a rotation-secured manner, for instance, by means of a recess in the guide part, in the direction of the longitudinal axis, with a pin-type guide.

The spindle is operatively connected to a rotary handle such that the slide is adjustable inside the guide part in the direction of the longitudinal axis of same. In other words, performing a rotational movement by means of the rotary handle causes a rotational movement of the spindle. A longitudinal threaded bore is provided in the slide, the internal thread of the slide being complementary with respect to the external thread of the spindle. The rotational movement of the spindle is transmitted by the above-described threads to the slide, resulting in the desired longitudinal displacement of the slide.

The slide furthermore includes means for the clamped attachment of the actual bone cerclage.

According to the invention, the means for clamping are configured as a specific assembly group, in particular a two-part assembly group. This assembly group is formed of an upper and a lower clamping piece. The upper clamping piece and the lower clamping piece are connected to each other by a hinged joint so as to be foldable.

A clamping screw is provided on the side opposing the hinged joint so as to exert a force on the clamping pieces. This exertion of force ensures that the bone cerclage located between the clamping pieces are fixed in position, the surfaces of the clamping pieces pointing to the bone cerclage having special clamping structures that permit the fixing both of wire cerclages and band or rope bone cerclages in position.

In a preferred embodiment of the invention a surface for attachment to the slide is formed on the lower clamping piece. This surface is capable of sliding in a longitudinal recess of the guide part, and may serve to improve the positional stability. In a useful embodiment the surface provided on the clamping piece is attached to the slide by screwing.

In an embodiment of the invention the clamping screw is mounted on the lower clamping piece captively and pivotably, and can be pivoted into a recess on the upper clamping piece. Thus, upon pivoting the clamping screw out of the recess, the hinged joint between the clamping pieces can be opened and the cerclage can be introduced. A subsequent pivoting movement of the clamping screw into the recess on the upper clamping piece, and fixing the clamping screw by means of a knurled nut, causes the required force to act on the bone cerclage located between the clamping pieces.

As was already mentioned before, the guide part may be configured as a tube having a longitudinal recess for the slide and clamping piece guidance. Furthermore, a guide rail for the bone cerclage is preferably provided at the end of the guide part opposite the rotary handle.

This guide rail may have a rail-type groove which is adapted to the width of the band cerclage to be maximally used. In order to prevent the respective bone cerclage from jumping out of the guide rail, it is possible to cover same with a clip or web.

Moreover, in order to improve the operability of the tensioning device, it is possible to fix a handle to the guide part, the handle preferably extending perpendicular to the longitudinal direction of the guide part.

This handle may be fixed on the left or right side of the guide part, depending on left-handed or right-handed operation. This fixing may be accomplished by a screw connection, respectively, screwing a threaded pin into an internal thread on the guide part. To this effect, the handle includes a threaded bolt.

The surfaces of the clamping pieces pointing to the bone cerclage have, in one specific embodiment of the invention, a height offset such that, on clamping the bone cerclage, the cerclage is secured by a step formed by said height offset. Even if forces exerted on the cerclage by means of clamping screw, knurled nut and clamping pieces are relatively small, this measure permits holding the cerclage without slipping out.

In another configuration of the surfaces of the clamping pieces, extending in the longitudinal direction of the guide part, the surfaces of the clamping pieces are each provided with at least one, preferably several grooves extending substantially in parallel. These grooves serve to receive in particular a wire cerclage, respectively, rope cerclage. The widths between the upper and lower clamping pieces for receiving the wire or band cerclage, i.e. the depth of the grooves, are chosen such that also smaller diameter ropes and wires can be reliably fixed.

The above-explained clamping screw, which is operatively connected to a knurled nut, is improved such that the knurled nut has, in addition to the usual knurls for actuating the nut, a recess on the upper surface side serving to receive a tool. This recess can be, for example, a polygon socket for receiving a tool having an external polygon, thereby allowing, if necessary, to release, respectively, actuate the knurled nut more easily with the aid of the tool.

The invention will be explained in more detail below by means of an exemplary embodiment and with reference to the figures.

In the drawing:

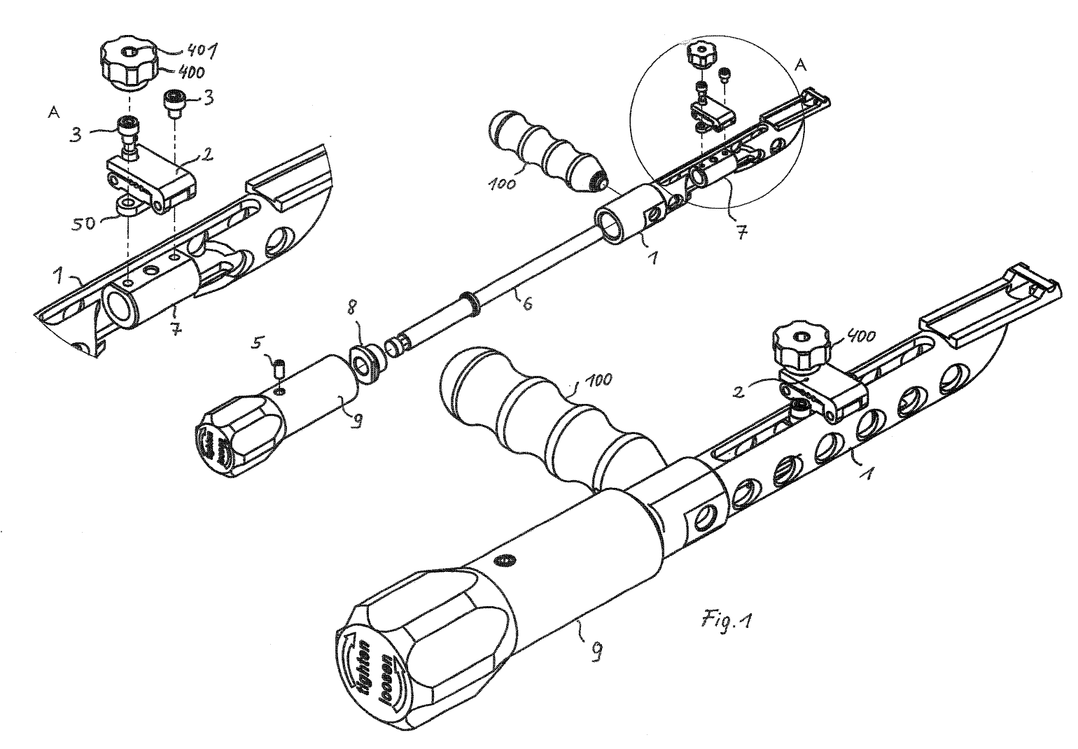

FIG. 1 shows an assembly drawing of the tensioning device according to an embodiment of the invention, as well as an exploded view and a detail drawing of the slide with an upper and a lower clamping piece;

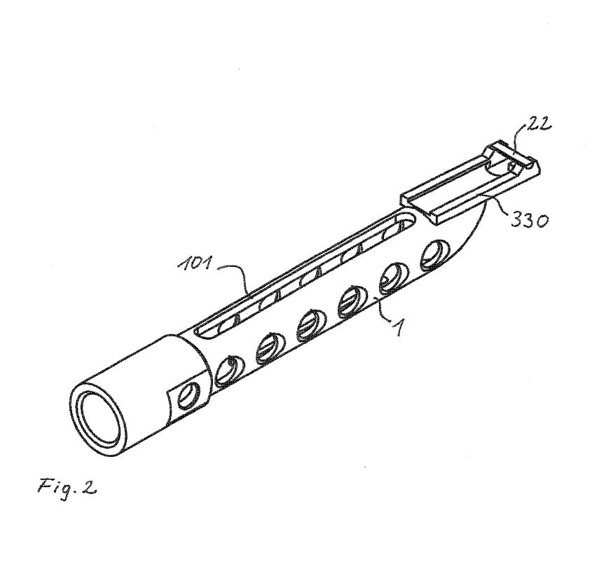

FIG. 2 shows a detailed view of the guide part, configured as a guide tube;

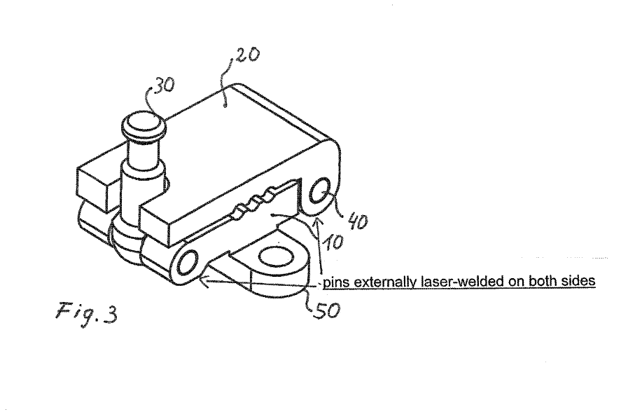

FIG. 3 shows a perspective detailed view of the clamping pieces in the folded, closed state (without knurled nut);

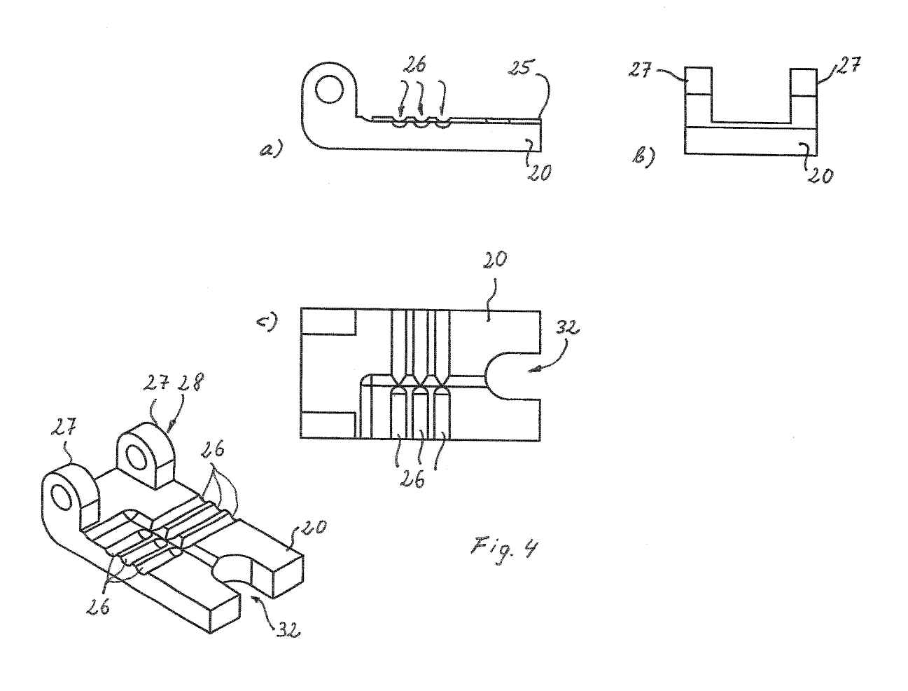

FIG. 4 shows a perspective view and a three-way view of the upper clamping piece; and

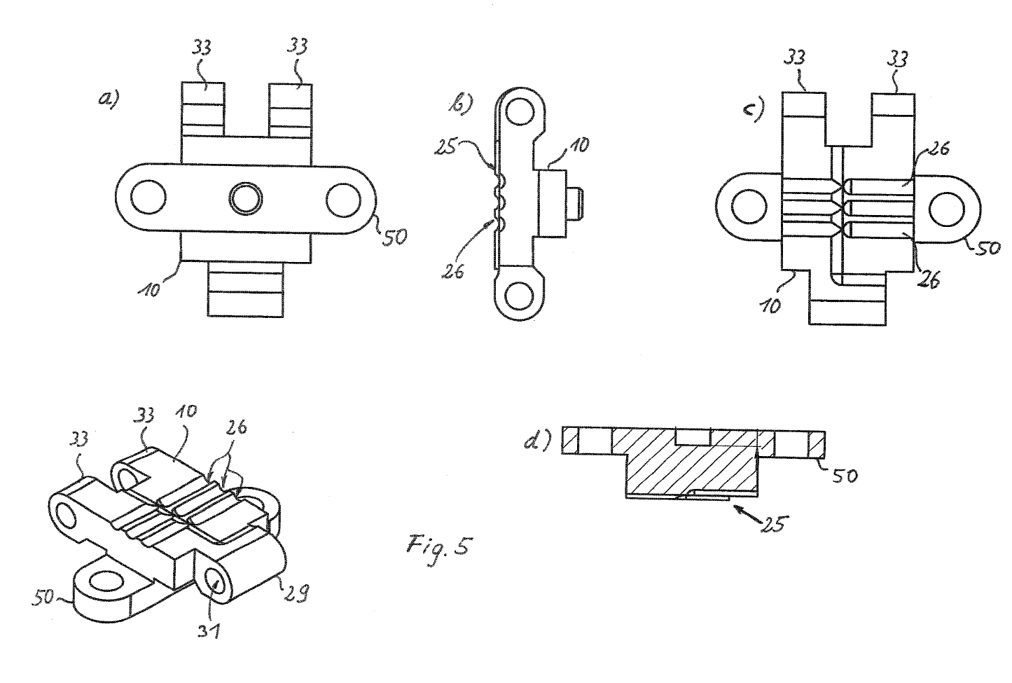

FIG. 5 shows a perspective view of the lower clamping piece and lateral views of same.

The dimensions specified in FIGS. 4 and 5 are of a pure exemplary nature and merely serve to illustrate a possible constructional embodiment of the parts according to the invention, without restrictions to the dimensions. Scaling in the sense of increasing and decreasing is within the scope of the invention and corresponds to a specialist modification.

As can be seen in the overall picture according to FIG. 1, the tensioning device is formed of a substantially tubular guide part 1 having a spindle 6 mounted rotationally movable in the interior thereof.

The spindle 6 takes along a slide 7 upon a rotational movement, the slide 7 being secured against rotation relative to the guide part 1.

The tensioning device furthermore comprises a rotary handle 9 which can be fixed, in a clamped fashion, at the end of the spindle 6 extending to the handle by a cylindrical pin 5.

A plug-like screw 8 is in communication with an internal thread of a respective end of the guide part 1 (also see FIG. 2). The through bore recognizable on part 8 of FIG. 1 serves the sliding guidance of the threadless end of the spindle 6.

Upon the performance of a rotational movement, using the rotary handle 9, the slide 7 moves in the longitudinal direction relative to the guide part 1.

A two-part assembly group 2, made of an upper 20 and lower clamping piece 10 (see FIG. 3), serves as means for clamping a bone cerclage not illustrated in FIG. 1.

By means of a fixing surface 50, which may be designed as a prolongation including bores, the assembly group 2 can be fixed to a corresponding surface of the slide 7 using a screw connection (screws 3).

The upper and lower clamping pieces 10; 20 are connected by a hinged joint 40 (FIG. 3) to be foldable. On the side opposite the hinged joint a clamping screw 30 (FIG. 3) is provided to exert a force on the clamping pieces 10; 20 and fix the bone cerclage (not shown) located between the clamping pieces 10; 20 in position. In this regard, a knurled nut 400 is used in combination with the clamping screw 30, which has a polygonal recess 401 on the upper surface side to apply there a corresponding tool having an external polygon and use same to actuate the knurled nut.

To allow a better handling of the tensioning device a handle 100 may be fixed to the guide part 1, e.g. by a screw connection. Depending on the left-handed or right-handed operation, this handle 100 may be inserted into a left-sided or right-sided threaded opening.

In a preferred embodiment the guide part 1 includes a longitudinal recess 101 for the slide and the clamping piece guidance. In addition, a guide rail 330 is provided, which has a groove that is adapted to the maximum width of a cerclage band. The guide rail 330 may additionally be covered by a clip 22 to prevent the respective bone cerclage from jumping out.

The two-part assembly group, as mentioned in FIG. 1, will now be explained in more detail by means of FIG. 3, showing a perspective view of the assembly group.

A lower clamping piece 10 cooperates with an upper clamping piece 20, the clamping pieces 10 and 20 being connected by the hinged joint 40. To this end, a pin defining an axis of rotation is used.

The screw 30 for exerting the clamping force between the lower and upper clamping pieces 10; 20 is mounted captively and pivotably on the lower clamping piece 10 and can be pivoted into or out of the recess located in the upper clamping piece 20 so as to allow an unobstructed unfolding and folding of the clamping pieces 10; 20. As can be seen in FIGS. 4 and 5, the surface sides of the clamping pieces 10 and 20, which point to each other, have a structuring that serves to reliably hold the clamped bone cerclage.

The lateral view according to FIG. 4a) shows that the surface structures have a height offset 25. This height offset 25 is also provided at the lower clamping piece 10 shown in the lateral view according to FIG. 5b).

The height offset of the lower clamping piece 10 is also recognizable, along lines B-B, in the sectional view according to FIG. 5d).

The respective height offset 25 between the clamping pieces 10 and 20 is realized by the interaction of a projection of one of the clamping pieces having a corresponding height with a recessed portion having a corresponding height, and vice versa, thus allowing the so realized step to fix the corresponding bone cerclage material reliably in position.

Additionally, the surfaces of the clamping pieces 10; 20 have several grooves 26 running in parallel, whose groove depth, respectively, radius is adapted to the material of the wire or rope cerclages to be received and clamped.

The position of the grooves can be seen in the lateral view and top view according to FIGS. 4a) and c), and the perspective view according to FIG. 4, which analogously applies to FIGS. 5, 5b) and 5c).

As can be seen in the illustrations of FIG. 4, the upper clamping piece 20 includes two projections 27 with a through bore 28 for receiving the axle 40, which also penetrates the lateral prolongation 29 of the lower clamping piece 10 and the through bore 31 thereof.

The U-shaped recessed portion 32, formed on the upper clamping piece 20, serves to receive the clamping screw 30, as shown in FIG. 3.

The clamping screw 30 is pivotably mounted on the lower clamping piece 10 by means of an axle and the prolongations 33.

1. Tensioning device for bone cerclages, consisting of a guide part (1) with a spindle (6) located in the interior which receives a slide (7) secured against rotation relative to the guide part (1), wherein the spindle (6) is operatively connected to a rotary handle (9) such that the slide (7) is adjustable inside the guide part (1) in the direction of the longitudinal axis of same, and further including means for the clamped attachment of the bone cerclage on the slide (7),
characterized in that
the means for clamping comprise a two-part assembly group (2) formed of an upper (20) and a lower (10) clamping piece, wherein the upper (20) and the lower (10) clamping pieces are connected to each other by a hinged joint (40) so as to be foldable, and on the side opposite the hinged joint (40) a clamping screw (30) is provided to exert a force on the clamping pieces (10; 20) and fix the bone cerclage located between the clamping pieces (10; 20) in position, the surfaces of the clamping pieces (10; 20) pointing to the bone cerclage having clamping structures (25; 26) that permit the fixing of rope, wire and band bone cerclages in position.

2. Tensioning device according to claim 1,
characterized in that
a surface (50) for attachment to the slide (7) is formed on the lower clamping piece (10).

3. Tensioning device according to claim 1,
characterized in that
the clamping screw (30) is pivotably mounted on the lower clamping piece (10) and can be pivoted into a recess (32) on the upper clamping piece (20).

4. Tensioning device according to claim 1,
characterized in that
the guide part is configured as a tube (1) having a longitudinal recess (101) for the slide and clamping piece guidance, and a guide rail (330) for the bone cerclage is provided at the end opposite the rotary handle (9).

5. Tensioning device according to claim 1,
characterized in that
a handle (100) can be fixed to the guide part (1).

6. Tensioning device according to claim 1,
characterized in that
the surfaces of the clamping pieces (10; 20) pointing to the bone cerclage have a height offset (25) such that, on clamping the bone cerclage, the cerclage is secured by a step formed by the height offset.

7. Tensioning device according to claim 1,
characterized in that
the surfaces of the clamping pieces (10; 20), extending in the longitudinal axis direction of the guide part (1), each have at least one groove, preferably several substantially parallel grooves (26).

8. Tensioning device according to claim 1,
characterized in that
the clamping screw (30) is operatively connected to a knurled nut (400).

9. Tensioning device according to claim 8,
characterized in that
the knurled nut (400) has a recess (401) on the upper surface side for receiving a tool.

10. Tensioning device according to claim 5,
characterized in that
the handle (100) can be fixed on the guide part (1) depending on left-handed or right-handed operation.
